(19) United States
(12) Patent Application Publication
(10) Pub. No.: US 2004/0007538 A1
(43) Pub. Date: Jan. 15, 2004

(54) Method and system for fluid purification using ultraviolet energy

(75) Inventor: Sittichai Siriphraiwan, Poway, CA (US)

Correspondence Address: FOLEY & LARDNER, 402 WEST BROADWAY, 23RD FLOOR, SAN DIEGO, CA, 92101

(73) Assignee: Tru Industries

(21) Appl. No.: 10/193,963

(22) Filed: Jul. 12, 2002

(51) Int. Cl. C02F 1/32

(52) U.S. Cl. 210/748

(57) ABSTRACT

A system and method for treating standing fluid is disclosed. The disclosed method includes providing a radiation source such as an ultraviolet (UV) energy source submerged within a volume of standing fluid, such as in a tank or reservoir. The UV energy source is caused to emit a predetermined amount of UV or other radiation on a periodic basis to help purify the fluid. The amount of energy is sufficient to destroy pollutants in the standing fluid at least within a pre-determined distance of the UV energy source. According to one example, radiation energy may be emitted repetitively from a UV lamp for about one hour per each 24 hours repetitively.

### BACKGROUND OF THE INVENTION

1. Field of the Invention

The invention relates to fluid purification systems. In particular, the present invention relates to a method and system for purification of fluids such as water.

2. Related Art

The information contained in this section relates to the background of the art of the present invention without any admission as to whether or not it legally constitutes prior art.

Several systems are known for purifying fluids such as drinking water. These systems may include fluid filter systems to filter out particles in the fluid.

Further, filter systems such as reverse osmosis systems may treat the fluid such as water as it is flowing. Thus, these systems may be capable of purifying drinking water, for example, as demanded or required by the consuming public. The rate at which these systems can treat the water is generally limited for all systems by, for example, the rate of flow of the water through purification filters or other such components.

If the demand exceeds the rate limit, either unpurified water is supplied or the system fails to meet the demand. Thus, storage tanks or reservoirs have been used to collect reserve purified water in the event that demand for purified water exceeds the rate limit of the purification system. Stored water, however, even if purified prior to storage, may become polluted or otherwise unsuitable for drinking as a result of bacteria that may grow in the water within the storage tank. The unwanted bacteria may be introduced to the reserve water through contact with ambient air.

There have been numerous attempts at purifying liquids such as water using ultraviolet radiation or other techniques. For example, reference may be made to the following U.S. Pat. Nos.: 6,403,030; 6,296,775; 6,264,888; 6,139726; 6,110,424; 6,099,735; 5,935,431; 5,900,212; 5,874,741; 5,545,335; 5,484,538; RE34,513; 5,227,053; 4,762,613; 4,752,401; 4,204,956; 4,184,076; 4,103,167; 4,017,735; and 3,971,947. However, none of them disclose a technique for purifying still fluids such as water stored in a reservoir to maintain previously treated fluids in a purified manner.

### BRIEF DESCRIPTION OF THE DRAWINGS

In the following, the invention will be explained in further detail with reference to the drawings, in which:

FIG. 1 is a pictorial diagrammatic view of one embodiment of a fluid purification system, illustrating it partially disassembled, according to one example of the present invention;

FIG. 2a is a top view of the fluid purification system of FIG. 1;

FIG. 2b is a cross-sectional elevational view of the fluid purification system of FIG. 1; and

FIG. 3 is a cross-sectional elevational view, similar to FIG. 2b, of the fluid purification system of FIG. 1, connected to a control system shown in block diagram, according to one embodiment of the invention.

### DESCRIPTION OF CERTAIN EMBODIMENTS OF THE INVENTION

The disclosed embodiments of the present invention provide a system and a method for treating standing fluid in, for example, a storage tank or reservoir. The disclosed method includes providing a radiation source such as an ultraviolet (UV) energy source submerged within a volume of standing fluid, and causing the energy source to emit on a periodic basis a predetermined amount of UV or other radiation to help purify the fluid. The amount of UV or other energy is sufficient to destroy pollutants in the standing fluid within a pre-determined distance of the energy source.

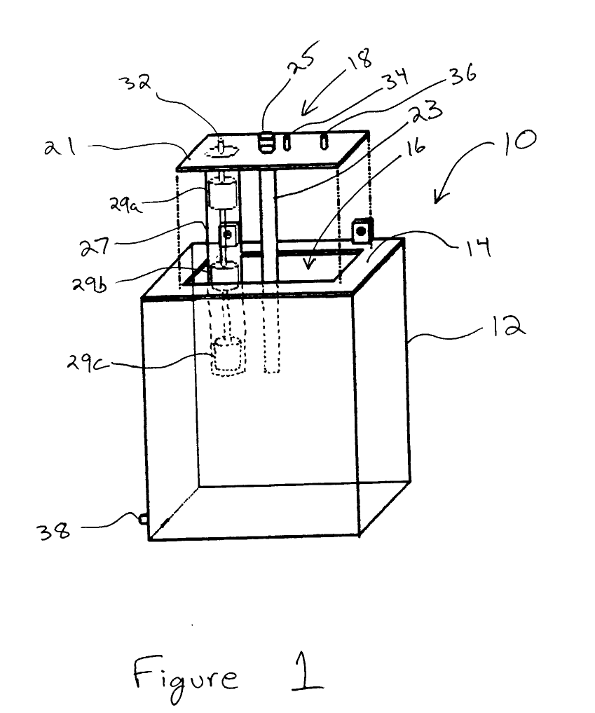

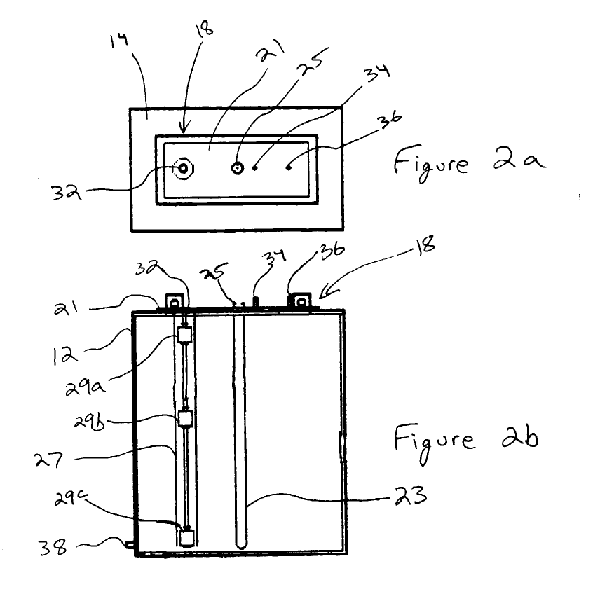

Referring now to the drawings, and more particularly to FIGS. 1, 2a and 2b thereof, this is a shown a fluid purification system 10 mounted in a storage tank or reservoir 12. In addition to storage tanks, systems according to the present invention may be mounted in reservoirs or any other containers capable of holding a volume of fluid.

In the preferred embodiment disclosed herein, the fluid being purified is water. However, it is to be understood that other fluid may be purified employing the principles of the present invention.

The storage tank 12 illustrated in FIG. 1 is rectangular in cross section. However, other configurations may be used as well. For example, a cylinder of circular or oval cross section may also be employed. Further, the orientation of the tank may be selected based on design choice. For example, the tank may be oriented either upright as shown, or resting horizontally on its side. Other configurations may also be used. In a preferred embodiment, the storage tank 12 is formed of stainless steel.

The storage tank 12 has a top surface 14 having an opening 16. The opening 16 is generally sufficiently large to accommodate the necessary. The inside of the storage tank 12 may be sized to store a volume of purified water that is sufficient to provide reserves in the event that instantaneous demand for purified water exceeds the rate limit of, for example, the flowing water from the purifier unit or feed system (not shown). In the disclosed embodiment, the tank 12 is hollow and is generally rectangular throughout its vertical axis. However, the tank 12 may be sized and shaped depending on the particular application.

The system 10 includes at least one ultraviolet (UV) purification module 18 for treating the fluid in the storage tank 12. The system 10 may include more than one UV module. The purification module 18 includes a panel 21 which covers over and seals the opening 16 in the top surface 14 of the storage tank 12 when assembled to the tank as shown in FIGS. 2a and 2b. The panel 21 may be sized slightly larger than the opening so that it may be secured to the top surface 14 by, for example, an industrial adhesive, bonding, or other affixing means may be employed.

A UV energy source 23 is attached to the underside of the panel 21. It is also contemplated to have two or more UV energy sources. According to the disclosed embodiment of the present invention, the UV energy source 23 is in the form of an elongated UV lamp, which is a submersible germicidal lamp. The source 23 is vertically and centrally disposed within the tank 12 as shown in FIG. 2b.

The UV energy source 23 as illustrated in FIG. 1 may be conventional, UV lamp which includes an outer elongated quartz sleeve. The UV lamp may be selected from conventional currently available UV lamps, or may be a customized lamp. For example, a UV lamp rated for a 5-year life or longer with one hour of use per day may be selected. This lamp's expected life may be longer if it is used for an extended period each day. For example, such lamps may have a rated lifetime of 10-15 years if used continuously or for longer intervals of time. However, due to the relatively low cost of the lamp, its use may be designed for use during shorter intervals of time to conserve electrical energy.

The UV radiation energy from the energy source 23 destroys certain pollutants such as certain bacteria or micro-organisms. The amount of energy required from the energy source 23 depends greatly on the amount of water to be treated and/or the size of the tank or reservoir. Typically, a dose of UV radiation of approximately 40,000 μWatts-second/cm² or more may be required to destroy the bacteria. The total amount of energy emitted by the energy source 23 would require that the necessary dose reach all portions of the interior surface of the storage tank. As an example, consider a tank that is a circular cylinder (not shown) having a circular cross-section with a radius of nine inches with a 4-Watt UV lamp located along the central longitudinal axis of the cylinder. Thus, the time required for the UV lamp to destroy the bacteria or other micro-organisms on the inner surface of the tank may be calculated as:

40,000 μW-sec/cm²=(4 W)(10⁶ μW/W)*(time)/{(9 inches)(2.54 cm/inch)}²time=5.2 seconds.

Thus, for tanks with larger or smaller diameters, or larger or smaller cross-sections, a longer or shorter exposure time may be required to kill all the bacteria or other unwanted micro-organisms.

Further, although the selected UV lamp may have a rated life of 10-15 years, the performance and energy output of the lamp may begin to degrade with use. For example, a germicidal UV lamp's output may degrade by 50% of its output as compared to its previous year output. Thus, a lamp that is capable of achieving the necessary dosage in only a few minutes in the first year may require nearly an hour after a few years of use.

Accordingly, in a preferred embodiment, the exposure time for the lamp may be set based on the lamp's anticipated performance in its final year. Thus, for example, the lamp may be set to emit UV radiation energy for one hour every day from the day of its installation to the day of its replacement. It is also preferred to activate the lamp wherever purified water is admitted to the tank 12. In such an embodiment, the dosage of UV radiation energy provided in the first few years may be substantially greater than that required, when the same periodic exposure time each year remains constant. However, little or no negative effects can be attributed to such overexposure. Thus, a simple implementation of the UV lamp may be achieved without the need to constantly update the exposure time necessary.

While a fixed or constant exposure time such as one from each 24 hours interval is presently preferred, it is contemplated that the exposure time may be varied or otherwise altered. For example, with a newer UV lamp, the exposure time may be shorter, and after a year or more, the exposure time may be adjusted to be longer to account for the degradation of the UV lamp.

Referring again to FIG. 1, the purification module 18 includes a UV energy source adapter 25 through which electrical power may be supplied to the UV lamp. Further, the power supplied to the UV lamp may be controlled through an on/off switch, for example, by a UV control circuit, as described below.

In certain embodiments of the invention, a level sensor 27 may be provided to detect the level of the fluid in the tank 12 or a reservoir. In the illustrated embodiment, the level sensor 27 includes three vertically aligned floats 29a, 29b, 29c, which are actuated by the presence of water at their respective levels. The floats are conventional and may be mounted within a stainless steel tube. The operation of floats is well known to those skilled in the art. The floats 29a, 29b, 29c generate signals that may be transmitted to a control circuit through a level sensor interface 32 provided on the panel 21.

The panel may also include a fluid feed inlet 34 for allowing fluid to be fed into the tank 12. Further, an air flow nozzle 36 may be provided to allow air to move in and/or out of the tank 12 as the fluid level in the tank 12 changes. In the illustrated embodiment, the storage tank 12 is provided with a fluid outlet nozzle 38 near the bottom of the tank. Flow of fluid out of the tank through the outlet nozzle 38 may be controlled by a control circuit.

FIGS. 2a and 2b illustrate an assembled purification module 18 and storage tank 12. As most clearly seen in FIG. 2b, the UV energy source 23 extends vertically from the top of the tank to substantially its bottom and is adapted to be disposed within the water of the tank 12 so that the water within the tank can be irradiated with UV radiation periodically. It will be understood by those skilled in the art that the orientation of the energy source 23 may be varied according to design choice. For example, in other embodiments, the energy source 23 may be oriented horizontally from the left end of the tank to the right end. The orientation of the energy source 23 should ensure that energy from the UV lamp reaches all portions of the tank 12.

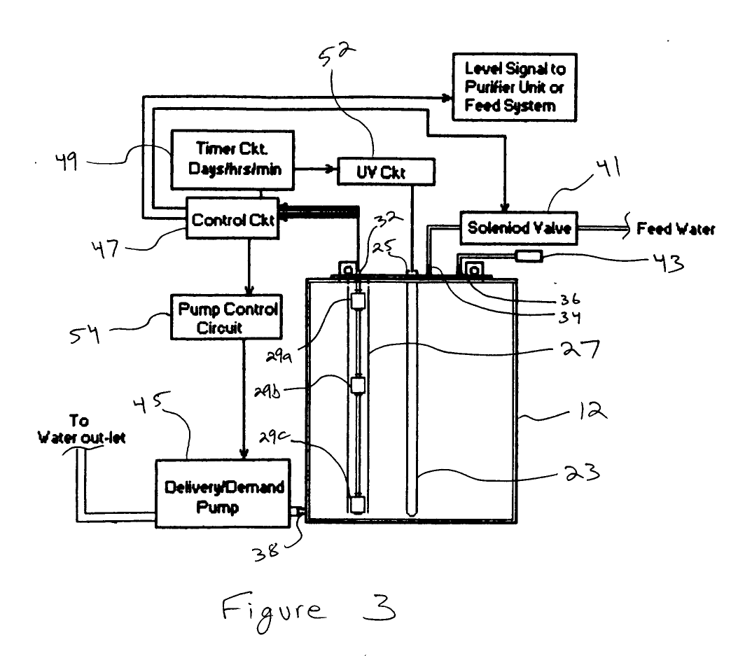

FIG. 3 illustrates the purification system of FIGS. 1-2b connected to a control system according to one embodiment of the invention. Fluid may be fed into the storage tank 12 through the fluid feed inlet 34 by way of, for example, a solenoid valve 41. Operation of the solenoid valve 41 may be controlled by a control circuit 47, which may receive inputs from various sources, including manual operator input.

As fluid is supplied into the tank 12, the air in the tank is allowed to be vented to the atmosphere through the air flow inlet 36. An air filter 43 may be connected to the air flow nozzle 36. However, the functionality of the air filter 43 may be required only when air is required to enter the tank, as described below.

A delivery/demand pump 45 may be provided to extract purified water from the storage tank 12 through the outlet 38 near the bottom of the tank 12. The pump 45 may operate under the control of a pump control circuit 54, which may receive inputs from the control circuit 47. For example, the control circuit 47 may detect that the floats 29a, 29b and 29c detect a level of the water within the tank 12 to be sufficiently low to require fresh water to be admitted to the tank 12. In this event, the control circuit 47 responds to signals from the floats via a level sensor interface 32 and may send a signal to the solenoid valve 41 to permit fresh purified fluid to be admitted to the tank 12 until the water level rises to a desired level where the signal from the floats terminates.

As fluid is extracted from the tank 12, air rushes into the tank 12 to replace the volume of the extracted fluid. The air rushes into the tank 12 through the air flow inlet 36. The air filter 43 may remove undesirable particles from the air entering the tank to prevent pollution or contamination of the purified water.

In one embodiment of the system, when water is added to the tank 12, the control circuit 47 causes a signal to be sent to a UV control circuit 52 to cause the UV energy source 23 to emit UV radiation energy, thereby causing irradiation of the fluid in the entire tank to disinfect the water stored therein.

The control circuit 47 may include a timer circuit 49. In certain embodiments, the timer circuit 49 may be separate from the control circuit 47. The timer circuit 49 may be used to transmit a signal to the UV control circuit 52 on a periodic recurring basis in order to cause the emission of UV radiation energy from the energy source 23 to irradiate the still water or other fluid stored in the tank. For example, as mentioned previously, the UV energy source 23 may be energized for about one hour during each 24 hour period of time repetitively. In addition, each time the UV energy source is energized, it will energize during the tank filling period and will continue for one hour after the tank is filled.

Thus, a desired level of purified fluid may be stored for extended periods of time, and the purity of the fluid may be maintained.

While particular embodiments of the present invention have been disclosed, it is to be understood that various different modifications and combinations are possible and are contemplated within the true spirit and scope of the appended claims. There is no intention, therefore, of limitations to the exact abstract and disclosure herein presented.

What is claimed is:

1. A method of treating standing fluid, comprising:
submerging a radiation source within a volume of standing fluid; and
causing said source to emit on a periodic basis a predetermined amount of radiation;
said amount being sufficient to destroy pollutants in said standing fluid within at least a pre-determined distance of said source.

2. The method according to claim 1, wherein said radiation is UV radiation and said amount of radiation is a predetermined UV radiation dosage at a furthest distance in said volume from said radiation source, wherein said energy dosage is calculated by:
W*T/d²,
where:
W is the wattage of said energy source,
T is the length of time said energy source emits energy, and
d is the furthest distance from the radiation source in said volume.

3. The method according to claim 2, wherein said UV radiation dosage is about 40,000 μWatts-seconds/cm² or more.

4. The method according to claim 1, wherein said periodic basis includes a predetermined length of time in about 24 hours.

5. The method according to claim 1, wherein said radiation source includes a UV lamp and an elongated quartz tube.

6. The method according to claim 1, wherein said volume of standing fluid is in a storage tank.

7. The method according to claim 6, wherein said storage tank is substantially cylindrical.

8. The method according to claim 7, wherein said storage tank is circular in cross section throughout its axial length.

9. The method according to claim 6, wherein said tank is composed of stainless steel.

10. A system for treating standing fluid, comprising:
a radiation source adapted to be mounted within a volume of standing fluid;
means for causing said radiation source to emit on a periodic basis a predetermined amount of radiation;
said amount being sufficient to destroy pollutants in said standing fluid within at least a pre-determined distance of said source.

11. The system according to claim 10, wherein said radiation is UV radiation and said predetermined amount of radiation is a predetermined UV radiation dosage at a furthest distance in said volume from said radiation source, wherein said energy dosage is calculated by:
W*T/d²,
where:
W is the wattage of said energy source,
T is the length of time said energy source emits energy, and
d is the furthest distance from the radiation source in said.

12. The system according to claim 11, wherein said UV radiation dosage is about 40,000 μWatts-seconds/cm² or more.

13. The method according to claim 10, wherein said periodic basis includes a predetermined length of time in about 24 hours.

14. The system according to claim 10, wherein said radiation source includes a UV lamp and an elongated quartz tube.

15. The system according to claim 10, wherein said volume of standing fluid is in a storage tank.

16. The system according to claim 15, wherein said storage tank is substantially cylindrical.

17. The system according to claim 16, wherein said storage tank is circular in cross section throughout its axial length cylinder.

18. The system according to claim 15, wherein said tank is composed of stainless steel.

19. The system according to claim 10, wherein said means for causing includes a control circuit and a timer circuit.
